(19) United States
(12) Patent Application Publication
(10) Pub. No.: US 2008/0190420 A1
(43) Pub. Date: Aug. 14, 2008

(54) Aerosol Medication Delivery Apparatus

(76) Inventor: Nimrod Lev, Savion GY (IL)

Correspondence Address: Medihale;Nimrod Lev, Havradim 1, Savyon GY, 56905, omitted

(21) Appl. No.: 11/908,865

(22) PCT Filed: Mar. 15, 2006
(86) PCT No.: PCT/IL06/00341

§ 371 Date: Sep. 17, 2007

Related U.S. Application Data
(60) Provisional application No. 60/660,346, filed on Mar. 11, 2005.

(51) Int. Cl. A61M 15/00

(52) U.S. Cl. 128/200.23

(57) ABSTRACT

The invention provides an aerosol medication delivery apparatus for use with a pMDI, wherein the pMDI has a discharge orifice from which the medication and propellant can be discharged forming an aerosol. The invented apparatus has a chamber housing having an inlet end and an outlet end and defining an interior space, wherein the inpui end receives the medication discharged from the discharge orifice of the pMDI into the interior space and wherein the medication can be withdrawn from the interior space by inhalation of a patient from the outlet end. The aerosol medication delivery apparatus also includes a valve at the inlet end. The valve is designed in a way that its perimeter surfaces mates with the pMDI internal and external perimeter surfaces when such is inserted, hence sealing it in a way that allows medication to flow freely through the internal cavity of the valve in the natural flow direction but prevents backflow into the pMDI housing and to the open air.

### TECHNICAL FIELD AND BACKGROUND OF THE INVENTION

The present invention is directed to the delivery of inhalant medications to subjects having pulmonary related diseases, and is particularly directed to the delivery of drugs from pressurized Meter Dose inhalers to persons with coordination problems (i.e. children aid the elderly).

Conventionally, inhalant-based medications such as aerosolized medications can be delivered in several ways. For example, some subjects directly insert a pressurized metered dose inhaler (“pMDI”) into their mouth, where the lips and mouth are able to substantially seal about the end of the pMDI device, and by pressing the pMDI head and inhaling in the same time, the medication is delivered to the patient's lungs.

Parts of the population, such as children and the elderly have difficulties to press and inhale in the same time, and the drug is wasted on oral tissues or into the ambient. In an attempt to overcome such a deficiency, others position a mask connected to a spacer that is connected to an inhalant aerosolized pMDI (such as AeroChamber by Trudell Medical or NebuChamber of AstraZeneca, connected to a pMDI) over the opening to deliver the medication into the opening.

Unfortunately, the mask can be somewhat cumbersome and/or inconvenient to use, With the above the mask has to be pressed towards the patient's face in order to create vacuum during inhalation, in order to open check-valves that prevents “back flow” of the sprayed medication to the ambient, as described in U.S. Pat. No. 6,371,117 and U.S. Pat. No. 6,557,549.

Furthermore, the currently used valves cause drug loss due to its obstructing behavior. Furthermore, spraying medication from the pMDI directly into the opening can cause premature deposit of the medication and impede proper delivery. The delivery shortcomings can, in turn, reduce the therapeutic treatment efficacy or value of the drug and potentially introduce undesirable effects to the patient.

Furthermore, tighten mask requests methods for safe exhalation in order to assure that no residues of exhaled breathing gases such as CO and CO2 remains inside the mask and can be re-breathed. Such method we can see in U.S. Pat. No. 5,645,049 that describes “exhalation valve for face mask with spacer chamber connection”.

Furthermore, is certain cases, a mouthpiece replaces the mask, as seen in U.S. Pat. No. 6,435,177 “Aerosol medication delivery apparatus and system” in order to create better valve-opening-vacuum during inhalation.

The only published application that describes a spacer without front check-valve, US patent application 20050081850, “Spacer device” describes a rigid, non flexible, hollow rear portion that is attached to the pMDI's outlet in a manner that it is tighten to the internal surface of the pMDI's cover and prevents some aerosol to flow backwards during exhalation.

Furthermore, most rear portions of such spacers are designed only to hold the pMDI when its outlet is inserted into this rear portion, as described in U.S. Pat. No. 5,848,588. “Backpiece for receiving an MDI adapter in an aerosolization spacer”, and not to prevent back flow of aerosolized medication due to exhalation of the patient.

### DISCLOSURE OF THE INVENTION

The described invention provides an aerosol medication delivery apparatus for use with a pMDI canister having medication and a propellant contained therein under pressure, wherein the pMDI canister has a discharge orifice from which the medication and propellant can be discharged forming an aerosol.

The apparatus has a chamber housing having an inlet end and an outlet end and defining an interior space, wherein the inlet end receives the medication discharged from the discharge orifice of the pMDI canister into the interior space and wherein the medication can be withdrawn from the interior space by normal inhalation by a patient from the outlet end. The aerosol medication delivery apparatus also includes a valve at the inlet end. The valve has a valve seat and a valve member. The valve seat has a sealing surface and the valve member has a central open area and a sealing portion at the perimeter of the open area that mates with the pMDI when such is inserted. Due to its shape and to the tight contact to the pMDI portions, the valve allows medication to flow freely through it in one direction but prevents backflow into the pMDI housing and to the open air.

Since the valve allows medication to flow freely through it in the “inhalation direction”, there is no need to apply force on it in order to open it and hence there is no need to create vacuum through inhalation from tighten mask.

It is therefore a first object of the present invention to provide a more effective and/or relatively convenient method of administering inhalable medications via oral or nasal cavities of the patient.

It is yet another object of the present invention to provide an improved manner of delivering aerosolized medications through an intermediating spacer.

It is an additional object of the present invention to provide less irritating, safer, periodic, inhalation-based drug deliveries through oral or nasal cavities of the patient.

It is another object of the present invention to provide a device which can allow drug deliveries without the need of check valves.

These and other objects are satisfied by the present invention by an adapter which is in fluid connection with a conventional oral inhalation-based medication dispensing device and which is configured to fit oral or nasal cavities of the patient, without an internal check valve.

Preferably, the device alignably engages with the oral or nasal openings in the subject's face and substantially allows more effectively deliver aerosolized medications therein. The adapter is particularly suitable to operate with an aerosolized drug delivery device such as pMDI.

A first aspect of the invention is directed to an Aerosol medication delivery apparatus having a medication dispensing exit flow port and a drug flow path associated therewith for use with a subject having pulmonary related disease. The Aerosol medication delivery apparatus includes a hollow body having opposing first and second end portions and defining a flow path therebetween. The hollow body first end is configured as a shaped tubular cylinder that penetrates a (relatively short) depth into the outlet of any conventional pMDI and to seal it in a manner that will allow aerosol to flow in one direction but not in the opposite direction. The hollow body second end is located external to the user facial openings (mouth and nose) and is configured to cover the mouth and nose without a tighten touch.

An additional aspect of the present invention is a method of using an Aerosol medication delivery apparatus for pressurized Meter Dose Inhalers. The method includes the steps of providing an Aerosol medication delivery apparatus having first and second end portions and an internal flow path therebetween to a patient who needs aerosol medication administration; sliding the Aerosol medication delivery apparatus first end portion onto the pMDI; unfolding the adapter second end mask; activating the inhalation based medication dispensing device to release the inhalant medication; and directing the inhalant medication into the Aerosol medication delivery apparatus and then into the user inhalation upper airways through the mask or a mouthpiece in a inner that it is oriented generally downwardly toward the user's pulmonary region or lower airway.

The present invention allows patients that have to take medications through inhalation to more effectively use conventional and/or existing pMDIs by configuring the Aerosol medication delivery apparatus to sealably engage with the mouthpiece of same and providing an easy to use (in a preferred embodiment it is configured such that it can be easily slid onto the conventional devices) chamber which allows the patient to inhale the aerosol without tight attaching of the mask to his or her lace. By configuring easy to use adapters which can conveniently engage with off-the-shelf inhalers, certain subjects with coordination problems can more effectively employ inhalers directed to conventional oral (mouth) operation.

The Aerosol medication delivery apparatus within this application is different from similar spacers by its designed shape, its integral foldable mask, the rear cheek-valve and internal baffle plate.

The foregoing and other objects and aspects of the present invention are explained in the specification set forth below.

### BRIEF DESCRIPTION OF THE DRAWINGS

The Aerosol medication delivery apparatus for pressurized Meter Dose Inhalers is described in the attached drawings, where it can be seen, in FIGS. 1 and 2 that the device (1) has a hollow body (6) with inlet (10) and outlet (4) that leads to a mask (2) that can be folded around hinge (3). The Meter Dose Inhaler (20), when inserted to the device inlet (10), is sealed from its external side by seal (7) and from its internal side by seal (9). When the Meter Dose Inhaler canister (21) is pressed down, a dose amount is released as aerosol through the Meter Dose Inhaler outlet (22) to the device's inlet (10), where it passes through the internal seal (9) passage (8) into the hollow body (6). The flow is restrained by a fixed baffle plate (5) in order to keep most of the aerosol inside the hollow body (6).

As shown in FIG. 5, when the user (100) inhales, the pressure difference creates a flow of the aerosol from the hollow body (6) through outlet (4) into the mask (2) that covers the nose and the mouth of the user (100) and to the user airways.

FIG. 3 and FIG. 4 shows 2 possible designs for the rear seal;

FIG. 3 describes seal (60) which is made of 2 rubber parts, internal seal (63) and external seal (62). The internal seal (63) is based and hold by bracket (61) which is a part of body (6). Optional ventilation check-valves (64) are used to prevent vacuum effect inside the device during patient's inhalation by allowing ambient air to pass through it into the device.

FIG. 4 describes seal (70) which is made of one rubber part, where the external seal (71) and internal seal (72) are shaped as one unit. Optional ventilation check-valves (73) are used to prevent vacuum effect inside the device during patient's inhalation by allowing ambient air to pass through it into the device.

FIG. 5 describes how patient (100) is using the device (1) in combine with pMDI (20).

FIG. 6 describes the device (1) when a mouthpiece (150) with a channeling tube (160) are used instead of facial mask.

FIG. 7 describes the device (1) when a mask (2) is used with a channeling tube (160).

FIG. 8 and FIG. 9 gives a perspective view of the entire described device.

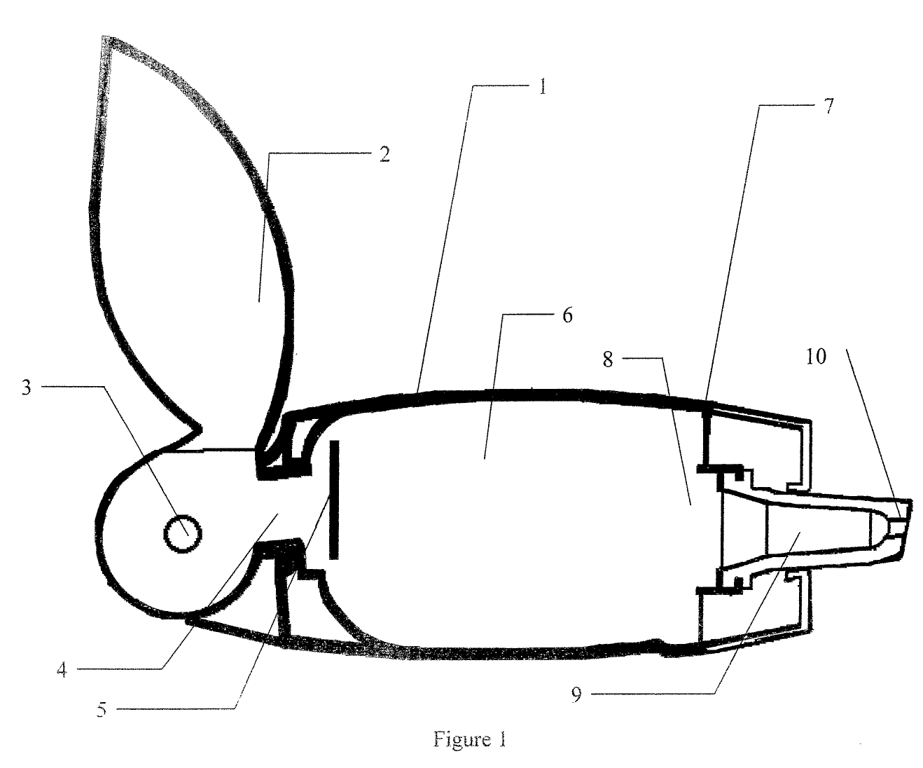

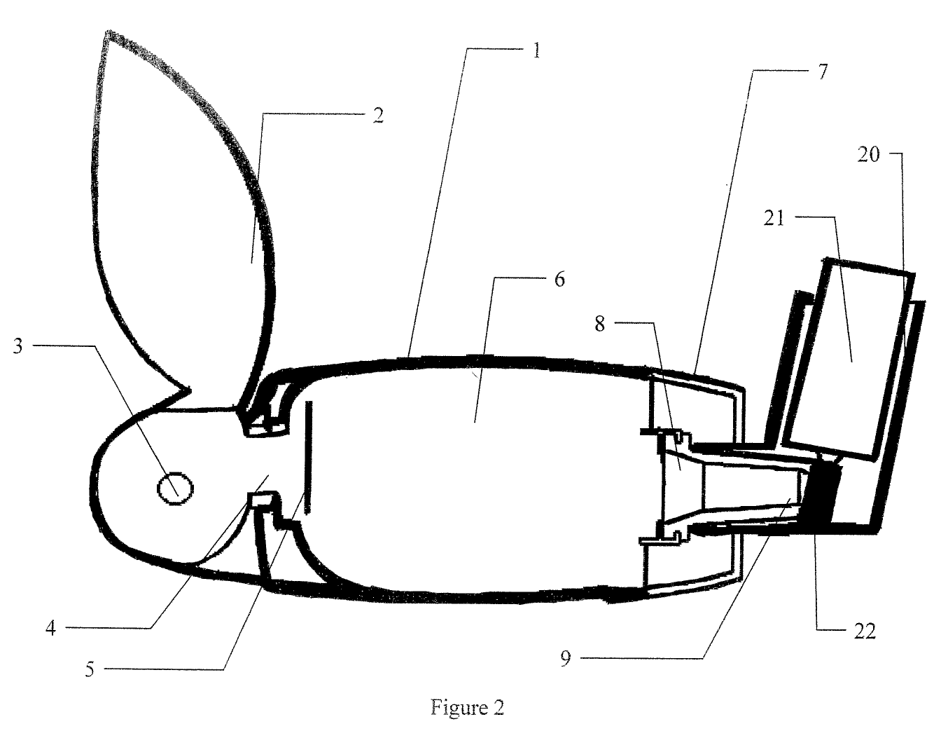

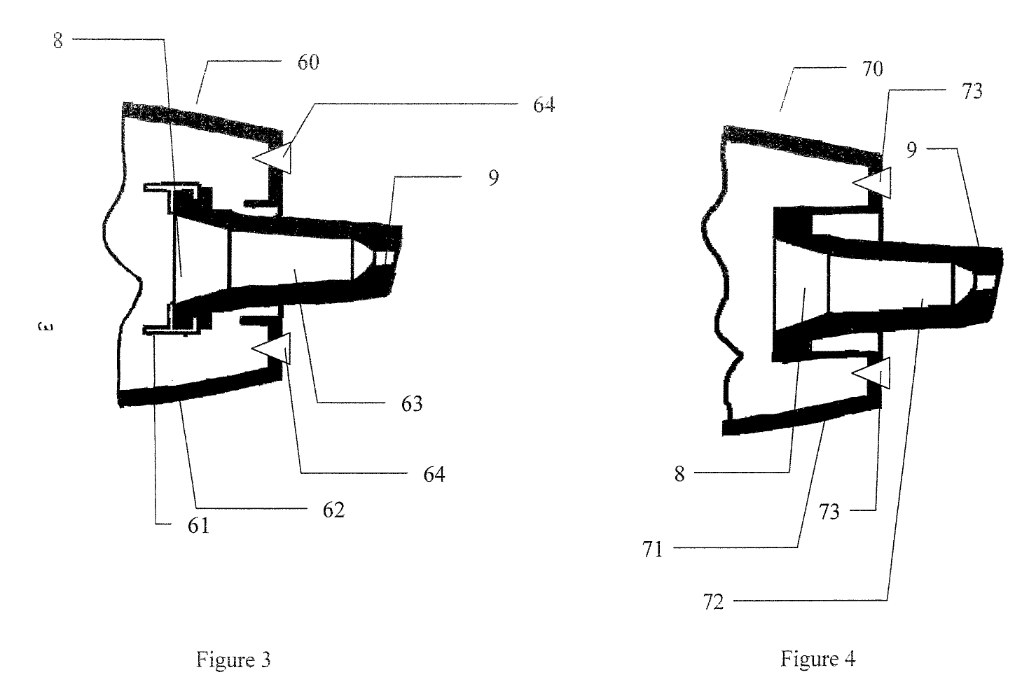

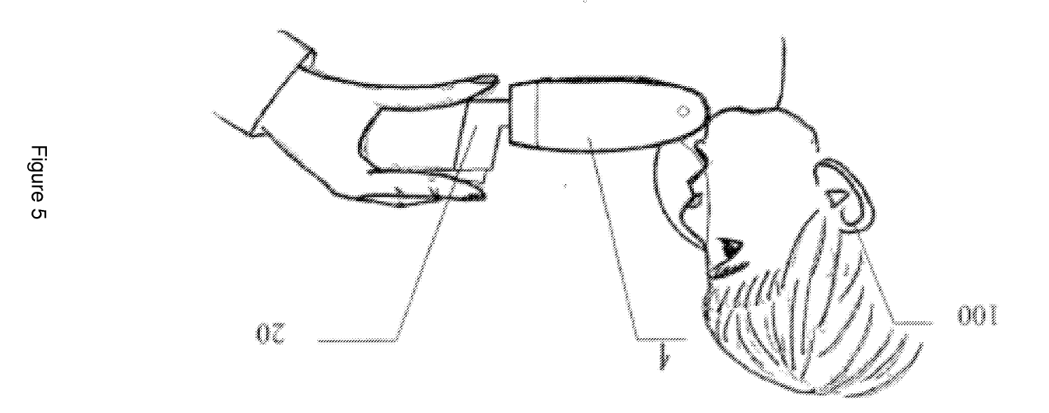

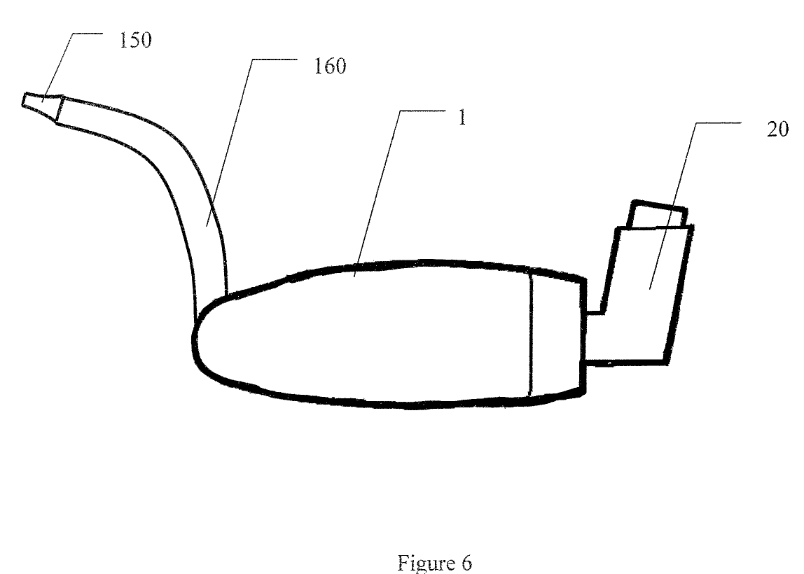

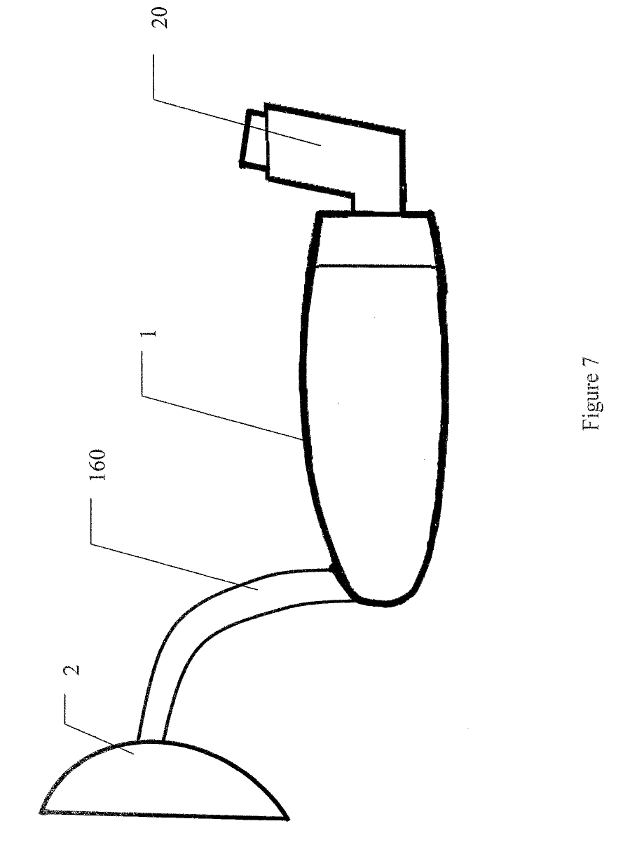

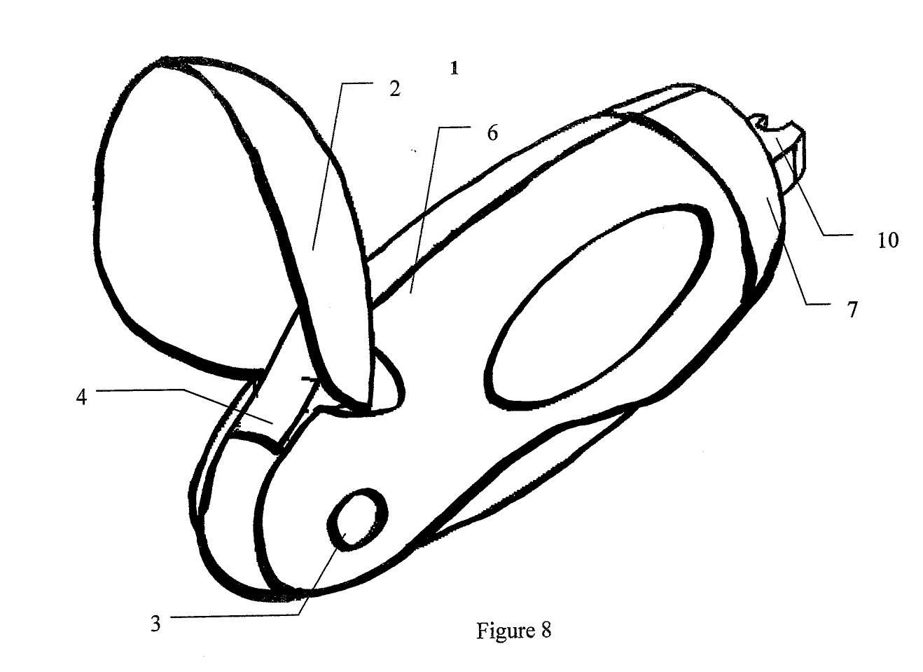

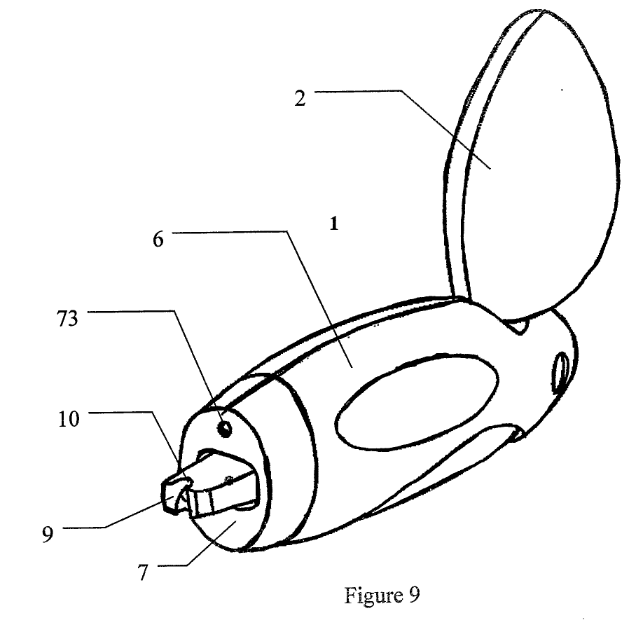

### BEST MODE FOR CARRYING OUT THE INVENTION

In order to carry out the invention, the best way is to manufacture the rigid parts of the device from medical plastic (i.e. plastic material that stand with medical standards) by injection molding and to manufacture the flexible parts (rear portion that acts like seal and unidirectional valve and front seal between rotating rigid parts) from medical rubber (such as silicon rubber) by injection molding as well.

Assembling these parts will be done using human skills in a simple manners as shown in the drawings.

The needed in vivo and in vitro tests, local regulation and administration will take part in order to place this apparatus as an alternative Aerosol medication delivery apparatus to currently used devices.

As shown in FIG. #5, a patient can use the device in combine with pMDI by simply inserting the pMDI into the device inlet, rotating the swivel mask to the desired position, pressing the pMDI in order to release aerosol medication dose and freely inhaling from the mask.

What is claimed is:

1. An aerosol medication delivery apparatus for use with a pressurized metered-dose inhaler (pMDI) having medication and a propellant contained therein under pressure, wherein the pressurized metered-dose inhaler has a discharge orifice from which the medication and propellant can be discharged forming an aerosol, the apparatus comprising:
a chamber housing having an inlet end and an outlet end and defining an interior space; and
a flexible valve located at the inlet end comprising a valve seat and a valve member, the valve seat having a sealing surface formed to fit outer perimeter of the intended pMDI, and the valve member having a central open channel and a sealing portion at a perimeter that mates with internal surface of intended pMDI parts, where said valve allows free flow of aerosol from said pMDI in the flow direction and prevents opposite flow when such flow is forced.

2. The aerosol medication delivery apparatus of claim 1, wherein a containment baffle is located before the outlet end, wherein the containment baffle includes a flat or convex surface; and the baffle surface faces downstream away from the input end of the chamber.

3. The aerosol medication delivery apparatus of claim 1, further comprising a mask which is connected to the apparatus through hinge that allows its rotation around defined axis.

4. The aerosol medication delivery apparatus of claim 1, wherein the valve portion is movable and can be disassembled from the apparatus and reassembled again.

5. The aerosol medication delivery apparatus of claim 3, wherein the mask is made of rigid material.

6. The aerosol medication delivery apparatus of claim 3, wherein the mask is made of flexible material.

7. The aerosol medication delivery apparatus of claim 1, wherein the central open area has oval cross section shape.

8. The aerosol medication delivery apparatus of claim 1, wherein the valve member and the outer valve portion are made as a single, non separable unit and forms a continuous surface.

9. The aerosol medication delivery apparatus of claim 1 wherein the valve member is separated from the outer valve portion.

10. The aerosol medication delivery apparatus of claim 3, wherein the mask Can Ire rotated around its axis in a manner that will allow the use of the medication delivery apparatus in several continuous positions of the apparatus body when said mask is placed on the patient's face.

11. The aerosol medication delivery apparatus of claim 3, wherein the apparatus is shaped and designed as a bird with a beak.

12. The aerosol medication delivery apparatus of claim 3, wherein the apparatus is shaped and designed in a figure that reminds an animal.

13. The aerosol delivery apparatus of claim 1, wherein the valve member is made of a rigid material.

14. The aerosol medication delivery apparatus of claim 1, further comprising a mouthpiece which is connected to the outlet of the apparatus in fluid connection.

15. The aerosol medication delivery apparatus of claim 1, farther comprising a mask and aerosol channel which is connected to the outlet of the apparatus in fluid connection.

16. A unidirectional valve, based on perimeter surfaces shape that can mate with shaped outlet of liquid or gas or aerosol generating system or device, with internal cavity that is free from obstructions.

17. The aerosol medication delivery apparatus of claim 1, further comprising a cheek valve in the unidirectional flexible valve that allow ambient atmosphere pressure equalization.
